(19) United States
(12) Patent Application Publication
(10) Pub. No.: US 2007/0189437 A1
(43) Pub. Date: Aug. 16, 2007

(54) General saddle cone beam CT apparatus and three-dimensional reconstruction method

(75) Inventors: Haiquan Yang, Kanagawa (JP); Meihua Li, Kanagawa (JP); Kazuhito Koizumi, Kanagawa (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: UNI-HITE SYSTEM CORPORATION, Yamato-shi (JP)

(21) Appl. No.: 11/657,027

(22) Filed: Jan. 24, 2007

(30) Foreign Application Priority Data
Jan. 25, 2006 (JP) 2006-16975

(51) Int. Cl. H05G 1/60; A61B 6/00; G01N 23/00; G21K 1/12

(52) U.S. Cl. 378/4

(57) ABSTRACT

A practical X-ray CT reconstruction method with a general saddle which overcomes restrictions in practical applications. A general saddle CT reconstruction method comprises: (a) placing an object relatively to a general saddle defined by the following conditions; (b) moving the X-ray source relatively to the object along the general saddle and detecting projections of the object on the detector plane; (c) filtering the projection along a predetermined filtering plane which is parallel to one of two major axes of the general saddle; and (d) performing three-dimensional reconstruction of the object through backprojection. The saddle curve is defined as such if the saddle curve is defined as such if a simple smooth curve which is single-connected, bounded and closed satisfies the following conditions against a family of continuous parallel planes in a three dimensional space: (1) for any plane in the family of parallel planes, there exist four different intersection points between the plane and the closed curve; (2) the four intersection points form a rectangle; and (3) rectangles obtained for any two different planes in the family of parallel planes are parallel to each other.

### FIELD OF THE INVENTION

The present invention relates to an X-ray CT (Computed Tomography) method and apparatus, and more specifically to a three-dimensional reconstruction method, apparatus and program from general saddle cone beam projections.

### BACKGROUND OF THE INVENTION

X-ray CT (tomography) apparatus such as a circular cone-beam CT apparatus and a helical cone-beam CT apparatus as representative examples have been well known. Moreover, properties of standard saddle curve (may be termed “saddle”, too) have been studied and reported (Non-patent document 1), and CT with a saddle has been proposed (Non-patent document-2). However, each of these methods and apparatus has certain disadvantages, respectively. Therefore, a novel and practical method and apparatus are expected.

[Non-Patent Document 1]

G. L. Zeng, G. T. Gullberg, S. A. Foresti, “Eigen-analysis of cone-beam scanning geometries”, Proceedings of the 1995 Fully 3D Meeting (International Meeting on Fully Three-Dimensional Image Reconstruction in Radiography and Nuclear Medicine) p. 261-265

[Non-Patent Document 2]

J. D. Pack, F. Noo, H. Kudo, “Investigation of saddle trajectory for cardiac CT imaging in cone-beam geometry”, Proceedings of the 2003 Fully 3D meeting. Phys. Med. Biol. 49, 2317-36, 2004

[Non-Patent Document 3]

Katsevich, A., “A general scheme for constructing inversion algorithms for cone beam CT”, Int. J. Math. Math. Sci. 21, 1305-21, 2003

### SUMMARY OF THE DISCLOSURE

The disclosures of these references are incorporated herein by reference thereto.

There are the following problems for conventional CTs according to our analyses.

1. Circular cone beam CT: Since a circular trajectory does not satisfy cone beam data completeness conditions (Tuy's condition), the quality of the reconstructed image deteriorates.

2. Helical cone beam CT: Since it requires overscan, it has problems such as increase in the measurement time and increase in X-ray exposure of objects. Moreover, serial imaging of data is difficult, because the X-ray source does not return to the original position after one rotation.

3. Properties of standard saddles have been studied by Zeng et al (1995) for the first time (Non-patent document 1). Thereafter, merits and properties of Cardiac CT to diagnose heart disease with a saddle defined as a curve at the intersection of two surfaces s₁ and S₂ have been studied by Pack et al since 2003 (Non-patent document 2). However the strict definition of the saddle trajectory by Pack et al excludes many useful curved trajectories. Moreover, the shift-variant CB-FBP image reconstruction algorithm proposed by Pack et al is complex to implement. And it is difficult and requires much computation, because the filtering is not given by a one-dimensional convolution function.

Therefore, a CT scanner with a saddle trajectory has not been realized until now, because it has been very difficult to obtain CT images with a saddle trajectory without simple and effective reconstruction algorithms.

A reconstruction method desired from the general curved trajectories has been applied to trajectories such as helix, circle and arc, circle and line (Non-patent document 3). However application to the saddle trajectories has not been discussed, because it is very difficult to verify the weighting equations in this method, and it is also very difficult to implement the reconstruction algorithm with a simple weighting. Moreover, it is unclear whether the method is applicable to truncation (whether the object is within the sight of camera or not). Until now, there has been no paper and report concerning the reconstruction with this method for the saddle trajectory.

According to an aspect of the present invention, it is an object of the present invention to provide a practical CT method and apparatus, and more specifically a cone-beam CT method and apparatus with a general saddle curve (may be termed “saddle” too) which overcomes restrictions on saddles defined by Pack. According to another aspect of the present invention, it is an object to provide an accurate, effective and simple method and apparatus for three-dimensional reconstruction of objects from projections (especially from projection data obtained with a saddle). Also it is an object of the present invention to provide a program therefor.

According to an aspect of the present invention, there is provided a novel X-ray CT method and apparatus. In this X-ray CT method and apparatus, the X-ray source (relative) trajectory is a general saddle which satisfies the cone-beam data completeness condition (Tuy's condition). According to another aspect of the present invention, there is provided an accurate, effective and simple three-dimensional image reconstruction method for projection data obtained with this method or apparatus.

According to an aspect of the present invention, there is provided a new X-ray CT apparatus—a general saddle cone-beam CT (hereafter called “general saddle CT”) apparatus for CT imaging of short objects and partial CT imaging of long objects. In the general saddle CT apparatus, the X-ray source (relative) trajectory in the X-ray source—detection system is a general saddle which satisfies the cone-beam CT data completeness condition (Tuy's condition).

According to a first aspect of the present invention, there is provided a general saddle CT scan method (Mode 1) comprising:

(a) placing an object in association with a general saddle curve defined by the following conditions; and

(b) moving an X-ray source relatively to the object along the general saddle and capturing an X-ray projection of the object on a detector plane;

wherein the saddle curve is a simple smooth curve which is single-connected, bounded and closed, and satisfies the following conditions against a family of continuous parallel planes in a three dimensional space:

(1) for any plane in the family of parallel planes, there exist four different intersection points between the plane and the closed curve;

(2) the four intersection points form a rectangle; and

(3) rectangles obtained for any two different planes in the family of parallel planes are parallel to each other. (Mode 1)

According to a second aspect of the present invention, there is provided a general saddle CT scan and reconstruction methods (Mode 2), besides the above (a) and (b), further comprising:

(c) filtering the projection data along a predetermined filtering plane which is parallel to one of two major axes of the general saddle curve; and

(d) performing three-dimensional reconstruction of the projection through backprojection.

Hereafter, “the general saddle curve” or “the general saddle” means a curve defined later on.

According to a third aspect of the present invention, there is provided a general saddle CT scan apparatus (Mode 3). The general saddle CT scan apparatus comprises:

(a) a support unit which supports an object relatively to a general saddle curve defined by the conditions below;

(b) a driving unit for an X-ray source-object-detector system, which provides relative movement of an X-ray source and a detector plane along a general saddle curve relatively to the object; and

(c) a capture unit which detects projection data of X-ray radiated from the X-ray source and penetrated through the object;

wherein the general saddle curve is a simple smooth curve which is single-connected, bounded and closed, and satisfies the following conditions against a family of continuous parallel planes in a three dimensional space:

(1) for any plane in the family of parallel planes, there exist four different intersection points between the plane and the closed curve;

(2) the four intersection points form a rectangle; and

(3) rectangles obtained for any two different planes in the family of parallel planes are parallel to each other.

According to a fourth aspect of the present invention, there is provided a general saddle CT scan and reconstruction device (Mode 4), besides (a), (b) and (c) in the above third aspect, further comprising the following (d) and (e):

(d) a filtering unit that filters the projection data along a predetermined filtering plane which is parallel to one of two major axes of the general saddle curve; and

(e) a backprojection and reconstruction unit that performs reconstruction of the object through backprojection.

According to a fifth aspect of the present invention, there is provided a computer program product for implementing and executing the above methods (Modes 1 and 2) by a computer. (Mode 5) The computer program may be recorded on recording medium of any kind available and computer- or machine-readable.

There are provided preferred modes of the present invention in the following.

[Mode 6]

There is provided a general saddle CT scan and reconstruction method according to the second aspect (Mode 2), wherein the filtering is performed along an intersection line between the filtering plane and the detector plane.

[Mode 7]

The filtering plane Π(λ,\vec{θ}) is chosen to satisfy the following conditions in relation to a point \vec{α}(λ) of the X-ray source:

(1) the filtering plane passes through the point \vec{α}(λ);

(2) the filtering plane is parallel to a vector \vec{θ}=(θₓ,θ_{y},θ_{z})^{T} extending from the point \vec{α}(λ) to a point on the detector plane; and

(3) the filtering plane is parallel to a vector \vec{ε}(λ,θ), where the vector \vec{e}(λ,\vec{θ}) is chosen to satisfy the following condition:

$$\vec{e} ( λ , \vec{θ} ) = \{ \begin{matrix}{\vec{e}}_{x} if {θ}_{z} \geq 0 \\ {\vec{e}}_{y} if {θ}_{z} < 0.\end{matrix}$$

[Mode 8]

The filtering is performed by the following equation:

$${g}_{F} ( λ , \vec{θ} ) = {\int }_{- π}^{π} ⅆ γ \frac{1}{sin γ} \frac{\partial }{\partial \overline{λ}} g ( \overline{λ} , ξ ( λ , θ , γ ) ) {\vert}_{\overline{λ} = λ} , θ ∈ {S}^{2}$$

$$where$$

$$\vec{ξ} ( λ , \vec{θ} , γ ) = cos γ \vec{θ} + sin γ \frac{\vec{e} ( λ , \vec{θ} ) - ( \vec{e} ( λ , \vec{θ} ) \cdot \vec{θ} ) \vec{θ}}{\vert \vec{e} ( λ , \vec{θ} ) - ( \vec{e} ( λ , \vec{θ} ) \cdot \vec{θ} ) \vec{θ} \vert} ,$$

and g(λ,\vec{ξ}) denotes the projection data.
[Mode 9]

The backprojection is performed by the following equation:

$$f ( \vec{x} ) = - \frac{1}{4 {π}^{2}} {\int }_{0}^{2 π} \frac{ⅆ λ}{\vert \vec{x} - \vec{a} ( λ ) \vert} {g}_{F} ( λ , \frac{\vec{x} - \vec{a} ( λ )}{\vert \vec{x} - \vec{a} ( λ ) \vert} ) ,$$

where f(\vec{x}) denotes CT value at each point of the object.
[Mode 10]

A well-designed general saddle curve defined in the following is employed as the general saddle curve, in case where the object is long, and the reconstruction region FOV is sandwiched between two (horizontal) planes crossing the object. The general saddle curve is termed “well-designed” subject to the following conditions:

(1) left (or right) part of a given general saddle curve above a first (horizontal) plane is located on the left (or right) side of the reconstruction region FOV;

(2) front (or back) part of a given general saddle curve below a second (horizontal) plane is located on the front (or back) side of a front (or back) plane of the reconstruction region FOV.

[Model 11]

A three-dimensional reconstruction of the object is performed by repeating the steps (c) and (d) for each value of the parameter λ of the general saddle curve.

[Mode 12]

The filtration comprises differentiating the projection data with respect to the parameter λ of the general saddle curve, weighting after the differentiation, interpolation before filtering, and 1D Hilbert transform of the interpolated data.

[Mode 13]

Filtration and coordinate transformation of the filtered data are performed after the 1D Hilbert transform.

[Mode 14]

The backprojection is performed on the filtered data after the 1D Hilbert transform.

[Mode 15]

The filtration is performed by defining two weighted data based on the projection data, and the reconstruction is performed using these data without differentiation operation with respect to the parameter λ of the general saddle curve.

[Mode 16]

The well-designed general saddle can be implemented by restricting value about one axis among two rotation axes in C-arm geometry or ring geometry with respect to a value of the other axis.

[Mode 17]

The general saddle curve is established by rotating the object (sample) about a rotation axis thereof, fixing either one of the X-ray source and the detector plane, and controlling their relative positional relationship.

[Mode 18]

The general saddle curve is established by fixing the X-ray source and the detector plane, rotating the object (sample) about its rotation axis and moving the object forward and reverseward (upward and downward) along the direction of its rotation axis in accordance with a predetermined equation.

The meritorious effects of the present invention are summarized as follows.

The general saddle CT apparatus according to the present invention provides a more (theoretically) accurate reconstruction of objects than the conventional circular cone-beam CT apparatus which does not satisfy the cone-beam CT data completeness condition. Since the general saddle CT apparatus eliminates the problem of overscan for CT imaging of a short object and partial CT imaging of a long object, it requires shorter measurement time and less X-ray exposure of objects than the helical cone-beam CT apparatus. Moreover, according to the present invention, an accurate, effective and simple three-dimensional image reconstruction method for the projection data obtained in the general saddle CT apparatus is provided. The reconstruction method is categorized to the FBP (filtered backprojection)-type reconstruction method in the same way as the FDK-type method which can be easily implemented. Therefore, the present invention provides the first easy-to-implement FBP-type reconstruction method, and solves problems concerning CT imaging with the general saddle X-ray source trajectory.

To solve problems in CT imaging of short objects and partial CT imaging of long objects, general saddles or saddle trajectories provide an X-ray source scan method for the CT apparatus according to the present invention.

[Definition of Terms]

The present invention employs novel concepts like “a general saddle curve (or trajectory)”, “a reconstruction region FOV (Field of View)”, “its front, back, left and right planes” and “a well-defined general saddle curve (or “saddle”). Therefore, these new concepts are defined in the following.

(1) The definition of a “general saddle curve (trajectory)”

The general saddle curve is a simple smooth curve which is single-connected, bounded and closed satisfies the following conditions against a family of continuous parallel planes in the three dimensional space:

(i) for any plane in the family of parallel planes, there exist four different intersection points between the plane and the closed curve;

(ii) these four intersection points form a rectangle; and

(iii) rectangles obtained for any two different planes in the family of parallel planes are parallel to each other.

(2) The definition of a reconstruction region FOV (field of view) and its front, back, left, and right planes

A reconstruction region FOV is defined as a part sandwiched (enclosed) between two (horizontal) parallel planes A and B when the object is long (e.g., shaped like a long column or cylinder).

A front plane of the reconstruction region FOV is the closest plane to the object in a set of vertical (orthogonal to the y-axis) planes in front of the object (in the negative direction of the y-axis).

A back plane of the reconstruction region FOV is the closest plane to the object in a set of vertical (orthogonal to the y-axis) planes on the back side of the object (in the positive direction of the y-axis).

A left plane of the reconstruction region FOV is the closest plane to the object in a set of vertical (orthogonal to the x-axis) planes on the left side of the object (in the negative direction of the x-axis).

A right plane of the reconstruction region FOV is the closest plane to the object in a set of vertical (orthogonal to the x-axis) planes on the right side of the object (in the positive direction of the x-axis).

Note that the terms “front, back, left and right” here are defined according to FIG. 1 with respect to the Cartesian coordinate system (x,y,z).

(3) Definition of a well-designed general saddle curve (trajectory)

A set of well-designed general saddle curves is defined according to the reconstruction region FOV and it is subset of a set of general saddle curves. In case where the object is long, and the reconstruction region FOV is sandwiched between two (horizontal) planes crossing the object, the general saddle curve is termed “well-designed” if it satisfies the following conditions:

(1) One part, i.e., left (or right) part of a given general saddle curve above the first (horizontal) plane (A) is located on the left (or right) side of the left (or right) plane of the reconstruction region FOV.

(2) One part, i.e., front (or back) part of a given general saddle curve below the second (horizontal) plane (B) is located on the front (or back) side of the front (or back) plane of the reconstruction region FOV.

### BRIEF DESCRIPTIONS OF THE DRAWINGS

FIG. 1 schematically shows a general saddle trajectory and a reconstruction region FOV.

FIG. 2 schematically shows the definition of a general saddle trajectory and its relation to the filtering plane and the detector plane.

FIG. 3 shows the relation between the coordinate for the general saddle trajectory and the coordinate for the projection data on the detector plane.

FIG. 4 shows the relation between the coordinate for the general saddle and the coordinate for the projection data on the detector plane for a C-arm geometry.

FIG. 5 shows an implementation example of a general saddle trajectory with a fixed X-ray source and a fixed detector.

FIG. 6 shows an implementation example of a general saddle trajectory with a fixed X-ray source and a movable detector.

FIG. 7 shows C-arm geometry which can be rotated about two major axes (λ,α).

FIG. 8 shows geometry for reconstructing a thin (or flat) object with a general saddle curve.

FIG. 9 shows a block diagram of an apparatus according to the present invention.

FIG. 10 shows results from Example 6. It shows cross sections of 3D reconstructed images by the VD algorithm and the VI algorithm using a Shepp-Logan phantom. The parameters for a standard saddle curve in Example 1 are R=12 cm, D=12 cm, h=4 cm, and the detector plane is at 7.2 cm.

FIG. 11 shows results from Example 7. It shows cross sections of 3D reconstructed images by the VD algorithm and the VI algorithm using a disc phantom. The parameters for general saddle curve in Example 1 are R=12 cm, D=12 cm, h=4 cm, and the detector plane is at 7.2 cm.

FIG. 12 shows results from Example 8. It shows cross sections of 3D reconstructed images by the VD algorithm and the VI algorithm using Shepp-Logan phantom. The parameters for C-arm geometry in Example 3 are R=7 cm, D=14 cm, k=0.3 cm, and the detector is at 7.2 cm.

### PREFERRED MODES OF THE INVENTION

1. The present invention employs a general saddle scan method. This is a novel X-ray CT method. A set of general saddle curves proposed in the present invention provides a more comprehensive set of curves which includes conventional sets of saddle curves and has the merit of applicability to Implementation of many kinds of hardware.

Note that the present invention includes a case where a combination of movement or rotation of the X-ray source, detector and the object has the same geometry as that of the general saddle trajectory in the image reconstruction space.

2. The present invention makes it possible to use a novel filtered backprojection reconstruction method (algorithm) in a general saddle CT apparatus. The reconstruction method has the merit of accuracy, high performance and easiness in implementation. The steps of the reconstruction method can be summarized in the following three steps.

Step 1: Differentiation of measured projection data along the general saddle curve.

Step 2: Filtering the differentiated projection data along a direction (a predetermined filtering plane) specified in the present invention.

Step 3: Backprojection of the filtered projection data.

The main feature of the reconstruction method (or algorithm) according to the present invention is that the filtering direction is specified on a plane which is parallel to one of two major axes (in the orthogonal coordinate system) of the general saddle curve.

3. According to the present invention, detailed steps for reconstruction algorithms are presented in accordance with each geometry for some various special examples of general saddles. Moreover, some modified algorithms which do not include differentiation along general saddle curves are also presented. These modifications are numerically more robust and reduce artifacts in the reconstructed images.

4. According to the present invention, hardware implementations of some special general saddle curves and their applicability are considered.

1. The Definition of a General Saddle Trajectory

[Definition]

Although the general saddle curve (trajectory) has been defined conceptually in the above “Definition of Terms” (1), it can be defined more exactly in mathematical terms as follows.

The “general saddle curve” is defined as such if a simple smooth curve C which is single-connected, bounded and closed satisfies the following conditions against a family of continuous parallel planes Π(\tilde{z})\tilde{z}ₘᵢₙ<\tilde{z}<\tilde{z}ₘₐₓ:

(1) for any \tilde{z}ₘᵢₙ<\tilde{z}<\tilde{z}ₘₐₓ, there exist four different intersection points between the plane Π(\tilde{z}) and a curve C, where the four intersection points are denoted as Aₖ(\tilde{z}),(k=1,2,3,4).

(2) Aₖ(\tilde{z}),(k=1,2,3,4) are continuous functions of parameter \tilde{z}, and Aₖ(\tilde{z}),(k=1,2,3,4) form a rectangle for any parameter \tilde{z}.

(3) rectangles obtained for any two different value of the parameter \tilde{z} are parallel. This means that the direction of A₁(\tilde{z}₁)A₂(\tilde{z}₁) and that of A₁(\tilde{z}₂)A₂(\tilde{z}₂) for any two different \tilde{z}₁ and \tilde{z}₂ are identical, and the same holds for A₂(\tilde{z}₁)A₃(\tilde{z}₁) and A₂(\tilde{z}₂)A₃(\tilde{z}₁) A₄(\tilde{z}₁) and A₃(\tilde{z}₂)A₄(\tilde{z}₂), A₄(\tilde{z}₁)A₁(\tilde{z}₁) and A₄(\tilde{z}₂)A₁(\tilde{z}₂), respectively.

In the present invention, the directions of A₁(\tilde{z})A₂(\tilde{z}) and A₂(\tilde{z})A₃(\tilde{z}) are termed “two major directions” of the general saddle. These major directions are independent of the value \tilde{z}.

2. Coordinate system and parameterization of the general saddle

2.1 A point O is the center of a rectangle A₁(\tilde{z}₀)A₂(\tilde{z}₀)A₃(\tilde{z}₀)A₄(\tilde{z}₀), where

$${\tilde{z}}_{0} = \frac{1}{2} ( {\tilde{z}}_{min} + {\tilde{z}}_{max} ) .$$

Here, a three dimensional space coordinate system with its origin at the point O is defined.

The unit vector \vec{e}ₓ along the x-axis is given by

$${\vec{e}}_{x} = \frac{{A}_{2} ( {\tilde{z}}_{0} ) - {A}_{1} ( {\tilde{z}}_{0} )}{‖ {A}_{2} ( {\tilde{z}}_{0} ) - {A}_{1} ( {\tilde{z}}_{0} ) ‖} ,$$

the unit vector \vec{e}_{y} along the y-axis is given by

$${\vec{e}}_{y} = \frac{{A}_{3} ( {\tilde{z}}_{0} ) - {A}_{2} ( {\tilde{z}}_{0} )}{‖ {A}_{3} ( {\tilde{z}}_{0} ) - {A}_{2} ( {\tilde{z}}_{0} ) ‖} ,$$

and the unit vector \vec{e}_{z} along the z-axis is given by \vec{e}_{z}=\vec{e}ₓ×\vec{e}_{y}.
2.2 In the coordinate system (x,y,z), the family of parallel planes Π(\tilde{z})\tilde{z}ₘᵢₙ<\tilde{z}<\tilde{z} can be parameterized by a parameter \tilde{z}. In the present invention, Π(\tilde{z}),\tilde{z}ₘᵢₙ<\tilde{z}<\tilde{z}ₘₐₓ denotes the family of parallel planes.
2.3 Using the Cartesian coordinate system (x,y,z), the general saddle trajectory C is parameterized as
\vec{α}(λ)=(αₓ(λ),α_{y}(λ),α_{z}(α)),0≦λ≦27π.
2.4 For a given value for parameter z, the parameterized general saddle trajectory C and Π(z) have four intersection points parameterized with λ as given by
\vec{α}(λ₁⁽⁼⁰⁾)=A₁, \vec{α}(λ₂⁽⁼⁾=A₂, \vec{α}(λ₃⁽⁼⁾)=A₃, \vec{α}(λ₄⁽⁼⁾)=A₄.

Moreover the following conditions are assumed:

$$\{ \begin{matrix}{a}_{z} ( λ ) \leq z & if & λ ∈ ( {λ}_{1}^{( z )} , {λ}_{2}^{( z )} ) ⋃ ( {λ}_{3}^{( z )} , {λ}_{4}^{( z )} ) . \\ {a}_{z} ( λ ) \geq z & if & λ ∈ ( {λ}_{2}^{( z )} , {λ}_{3}^{( z )} ) ⋃ ( {λ}_{4}^{( z )} , {λ}_{1}^{( z )} ) .\end{matrix}$$

In the present invention, x- and y-axes are called major axes of the general saddle.

2.5 In the present invention, we define a set of points as
Ω(C)={\vec{x}ε A̅₁̅A̅₂̅A̅₃̅A̅₄̅(z)|zₘᵢₙ<z<zₘₐₓ}.
where A̅₁̅A̅₂̅A̅₃̅A̅₄̅(z) is a set of inner and boundary points of the rectangle A₁A₂A₃A₄ for fixed value of z. We also define Ω⁰(C)={\vec{x}εΩ(C)\vec{x} is an inner point of Ω(C)}.

The set of points Ω⁰(C) satisfies the cone-beam data completeness condition (Tuy's condition). We assume that the reconstruction region filed of view FOV defined as
FOV={(x,y,z)|x²+y²≦B²,z^{b}ₘᵢₙ≦z≦z^{b}}
is a subset of Ω⁰(C).
<The Definition of a Well-Designed General Saddle Trajectory>

A general saddle is called well-designed relating to the FOV if it satisfies
α_{y}(λ)<−B if λε(λ₁⁼^{ₘᵢₙ}^{^{b}}⁾,λ₂⁽⁼^{ₘᵢₙ}^{^{b}}⁾),
α_{y}(λ)>B if λε(λ₃⁽⁼^{ₘᵢₙ} ^{^{b}}⁾,λ₄⁽⁼^{ₘᵢₙ}^{^{b}}⁾,
αₓ(λ)>B if λε(λ₂⁽⁼^{ₘₐₓ}^{^{b}}⁾,λ₃⁽⁼^{ₘᵢₙ}^{^{b}}⁾,
αₓ(λ)<−B if λε(λ₄⁽⁼^{ₘₐₓ}^{^{b}}⁾, λ₁⁽⁼^{ₘₐₓ}^{^{b}}⁾).
3. The set of general saddles includes ordinary saddles
3.1 A saddle trajectory is defined as the curve established at the intersection of two surfaces (S₁, S₂):
S₁={(x,y,z):z=s₁(x)} and
S₂={(x,y,z):z=s₂(y)}
where the two functions S₁(x), and S₂(y) satisfy the following conditions
s₁″(x)>0, s₁′(0)=0,s₁(0)<0 for any x,
s₂″(y)<0,s₂′(0)=0, s₂(0)=−s₁(0) for any y.
3.2 Since a family of planes Π(z),s₁(0)<z<s₂(0) parallel to the (x,y)-plane satisfies the conditions of the general saddle, the saddle trajectory defined above is a special example of the general saddles. All ordinary saddles are well-designed general saddles.
3.3 An example of a general but not ordinary saddle
Curve on the Sphere

A curve defined by (R cos(α(λ))cos λ,R cos(α(λ))sin λ,R sin(α(λ))^{T} where

$$α ( λ ) = {tan}^{- 1} \frac{k cos 2 λ}{\sqrt{1 - {k}^{2}}}$$

is an ordinary saddle when 0<k<0.5. However, the curve is not an ordinary saddle but a general saddle when 0.5≦k<1. Moreover, this curve is also a well-designed general saddle when 0.5≦k<1.
4. The definition of cone beam projection data

$$g ( λ , \vec{θ} ) = {\int }_{0}^{∞} ⅆ t f ( \vec{a} ( λ ) + t \vec{θ} ) , \vec{θ} ∈ {S}^{2} ,$$

where S² is the unit sphere.

5. The definition of a filter/correction backprojection operator
5.1 Differentiating

$${g}^{′} ( λ , \vec{θ} ) = \frac{\partial }{\partial λ} g ( λ , \vec{θ} ) .$$

5.2 Filtering

The filtering plane is composed of following three elements, i.e., the position of the X-ray source \vec{α}(λ), the point to be reconstructed \vec{x} and the direction \vec{e}. The filtering plane passes through a point \vec{α}(λ) and a point \vec{x}, and is parallel to a vector \vec{e}. We consider following two orthogonal vectors in the filtering plane,

$$\vec{α} ( λ , \vec{x} ) = \frac{\vec{x} - \vec{a} ( λ )}{‖ \vec{x} - \vec{a} ( λ ) ‖} , \\ \vec{β} ( λ , \vec{x} , \vec{e} ) = \frac{\vec{e} - ( \vec{e} \cdot \vec{α} ( λ , \vec{x} ) ) \vec{α} ( λ , \vec{x} )}{‖ \vec{e} - ( \vec{e} \cdot \vec{α} ( λ , \vec{x} ) ) \vec{α} ( λ , \vec{x} ) ‖} .$$

Any direction in the filtering plane can be parameterized by an angle γ from the vector \vec{α} and given by
\vec{θ}(λ,\vec{x},\vec{e},γ)=cos γ·\vec{α}(λ,\vec{x})+sin γ·\vec{β}(λ,\vec{x},\vec{e}).

The filtering operator is defined by

$${g}_{F} ( λ , \vec{x} , \vec{e} ) = {\int }_{- π}^{π} ⅆ γ \frac{1}{sin γ} {g}^{′} ( λ , \vec{θ} ( λ , \vec{x} , \vec{e} , γ ) )$$

This operator is essentially a one-dimensional (1D) Hilbert transform on the intersection line of the filtering plane and the detector plane.
5.3 The backprojection is applied to the filtered result after weighting.

$$K ( \vec{x} , \vec{e} , {λ}^{-} , {λ}^{+} ) = - \frac{1}{2 {π}^{2}} {\int }_{{λ}^{-}}^{{λ}^{+}} ⅆ λ \frac{1}{‖ \vec{x} - \vec{a} ( λ ) ‖} {g}_{F} ( λ , \vec{x} , \vec{e} )$$

If λ⁻>λ⁻, we regard (approximate) λ⁺ as λ⁺+2π in the above integration.

6. Reconstruction formula

For any point \vec{x}=(x,y,z₀)^{T} within the FOV, the plane z=z₀ and the general saddle trajectory intersect at four points \vec{α}(λ₁⁽⁼^{₀})⁾, \vec{α}(λ₂⁽⁼^{₀}⁾),\vec{α}(λ₃⁽⁼^{₀}⁾),\vec{α}(λ₄⁽⁼^{₀}⁾). Therefore the reconstruction formula at a point \vec{x}=(x,y,z₀)^{T} is given by

$$f ( \vec{x} ) = \frac{1}{2} \{ \begin{matrix}K ( \vec{x} , {\vec{e}}_{x} , {λ}_{1}^{( {z}_{0} )} , {λ}_{2}^{( {z}_{0} )} ) + K ( \vec{x} , {\vec{e}}_{y} , {λ}_{2}^{( {z}_{0} )} , {λ}_{3}^{( {z}_{0} )} ) + \\ K ( \vec{x} , - {\vec{e}}_{x} , {λ}_{3}^{( {z}_{0} )} , {λ}_{4}^{( {z}_{0} )} ) + K ( \vec{x} , - {\vec{e}}_{y} , {λ}_{4}^{( {z}_{0} )} , {λ}_{1}^{( {z}_{0} )} )\end{matrix} \} .$$

7. FBP-type implementation of the reconstruction method and its applicability to the axial truncation data

Superficially, the filtering plane is related to the point \vec{x}=(x,y,z₀)^{T} to be reconstructed in the above reconstruction formula and the point \vec{x}=(x,y,z₀)^{T} is needed in the filtering process. Therefore, it seems as if numerical implementation necessary for the reconstruction be very complex and totally different from the computation scheme of the FBP type.

However, in the well-designed general saddle, the filtering surface in the above reconstruction formula can be parameterized in one dimension as follows, in case where the projection angle λ is fixed.

Denote the intersection point between the filtering plane and the z-axis as (0,0,z)^{T}. A plane parallel to the x-axis is selected as a filtering plane if z>α₌(λ), and a plane parallel to the y-axis is selected as a filtering plane if z≦α₌(λ). This family of filtering planes is denoted by π(z).

From the definition of the coordinate for the general saddle trajectory, all filtering planes for the reconstruction formula are contained in the family π(z). Therefore the filtering can be performed prior to backprojection in the present invention. This filtering process is independent of the position of the point \vec{x}=(x,y,z₀) to be reconstructed and shares the same basic properties with the conventional FBP-type algorithms.

Also note that any plane in the family π(z) is not parallel to the z-axis for a well-designed general saddle trajectory, so the algorithm of the present invention is applicable to axial truncation data.

8. Special Example 1 (detailed reconstruction steps)

8.1 Definition

We consider a class of well-designed general saddles parameterized as
(R(λ)cos λ, R(λ)sin λ, α₌(λ))^{T},
where λ is the polar angle in the (x,y)-plane and the polar radius R(λ) satisfies R(λ)>0.
8.2 Detector Geometry

In the reconstruction space, the coordinate for the detector is given by

$$\{ \begin{matrix}{\vec{e}}_{u} ( λ ) = {( - sin λ , cos λ , 0 )}^{T} \\ {\vec{e}}_{v} ( λ ) = {( - cos λ , - sin λ , 0 )}^{T} \\ {\vec{e}}_{w} ( λ ) = {( 0 , 0 , 1 )}^{T}\end{matrix}$$

The detector plane is arranged to be orthogonal to the unit vector \vec{α}ᵥ(λ). The origin of the detector plane is placed at the projection of the point \vec{α}(λ) onto the detector plane. Cartesian coordinates (u,w) specify a pixel in the detector plane. The u-axis is parallel to the vector \vec{e}ᵤ, and the w-axis is parallel to the vector \vec{e}_{w}.

The distance between the detector plane and the X-ray source is denoted by D(λ)(>0), and the projection data on the detector plane is denoted by g_{f}(λ,u,w).

8.3 Reconstruction Steps

STEP 1: Filtering.

Each projection data g_{f}(λ,u,w) is modified/transformed into g_{f}^{F}(λ,u,w) according to the following filtering steps:
FF1: derivative at constant direction. Compute

$${g}_{1} ( λ , u , w ) = ( \frac{\partial }{\partial λ} + \frac{{D}^{′} ( λ ) u + {u}^{2} + {D}^{2} ( λ )}{D ( λ )} \frac{\partial }{\partial u} + \frac{{D}^{′} ( λ ) w + uw}{D ( λ )} \frac{\partial }{\partial w} ) {g}_{f} ( λ , u , w ) .$$

FF2: length-correction weighting. Compute

$${g}_{2} ( λ , u , w ) = \frac{D ( λ )}{\sqrt{{D}^{2} ( λ ) + {u}^{2} + {w}^{2}}} {g}_{1} ( λ , u , w ) .$$

FF3: interpolation before filtering. Compute
g₃(λ,u,z)=g₂(λ,u,w(u,z))
where z is a parameter for the filtering plane π(z), and w(u,z) is the intersection line between the filtering plane π(z) and the detector plane.
FF4: 1D Hilbert transform
Compute
g₄(λ,u,z)=∫_{-∞}^{∞}du′h_{H}(u−u′)g₃(λ,u′,z),
where h_{H} is the impulse response of the Hilbert transform.
FF5: Transform after filtering
Compute
g_{f}^{F}(λ,u,w)=g₄(λ,u,z(u,w)),
where z(u,w) denotes the parameter for the filtering plane which passes through the point (u,w) on the detector plane.
STEP 2: Backprojection

The filtered projection g_{f}^{F}(λ,u,w) is backprojected to formulate f at each point \vec{x}=(x,y,z) of the FOV according to the formula

$$f ( \vec{x} ) = \frac{1}{4 π} {\int }_{0}^{2 π} ⅆ λ \frac{1}{\vec{v} ( \vec{x} , λ )} {g}_{f}^{F} ( λ , \overline{u} ( \vec{x} , λ ) , \overline{w} ( \vec{x} , λ ) ) ,$$

where (\vec{u}(\vec{x},λ),\vec{w}(\vec{x},λ)) denote the coordinates of the intersection point of the detector plane and the line connecting the points \vec{x} and \vec{α}(λ), and
\vec{v}(\vec{x},λ)=R(λ)−x cos λ−y sin λ.

Note that by eliminating the procedure FF5 of STEP 1 in the above reconstruction steps and incorporating the corresponding procedure in the backprojection of STEP 2, resolution of the reconstructed image can be increased.

8.4 The above reconstruction step in 8.3 needs differentiation with respect to the parameter λ for the general saddle curve. The differentiation processing increases sampling and discretization errors, which are counted as drawbacks.

There are some modified formulae which eliminate the differentiation operation with respect to λ from the reconstruction formula at 8.3. Here, an example thereof is shown.

First, we define two weighted data by

$${\tilde{g}}_{f} ( λ , u , w ) = \frac{D ( λ ) + {uR}^{′} ( λ ) / R ( λ )}{\sqrt{{D}^{2} ( λ ) + {u}^{2} + {w}^{2}}} {g}_{f} ( λ , u , w ) , \\ and$$

$${{g}^{⋒}}_{f} ( λ , u , w ) = \frac{D ( λ )}{\sqrt{{D}^{2} ( λ ) + {u}^{2} + {w}^{2}}} {g}_{f} ( λ , u , w ) .$$

Then, the reconstruction formula is given as follows:

$$f ( \vec{x} ) = - \frac{1}{4 {π}^{2}} {\int }_{0}^{2 π} ⅆ λ \frac{D ( λ ) R ( λ )}{{\overline{v}}^{2}} \int \frac{ⅆ u}{{( \overline{u} - u )}^{2}} {\tilde{g}}_{f} ( λ , u , w ) {\vert}_{w = \overline{w} + κ ( u - \overline{u} )} - \frac{1}{4 {π}^{2}} {\int }_{0}^{2 π} ⅆ λ \frac{D ( λ ) ( κ R ( λ ) - {a}_{z}^{′} ( λ ) ) - {R}^{′} ( λ ) ( \overline{w} - κ \overline{u} )}{{\overline{v}}^{2}} \int \frac{ⅆ u}{\overline{u} - u} ( \frac{\partial }{\partial w} {{g}^{⋒}}_{f} ( λ , u , w ) ) {\vert}_{w = \overline{w} + κ ( u - \overline{u} )} - \frac{1}{4 {π}^{2}} {\int }_{0}^{2 π} \frac{D ( λ ) ( κ R ( λ ) - {a}_{z}^{′} ( λ ) ) - {R}^{′} ( λ ) ( \overline{w} - κ \overline{u} )}{{\overline{v}}^{2}} χ ( λ , \overline{u} ) \int ⅆ u ( \frac{\partial }{\partial w} {{g}^{⋒}}_{f} ( λ , u , w ) ) {\vert}_{w = \overline{w} + κ ( u - \overline{u} )} ,$$

where κ is the gradient of the intersection line between the filtering plane and the detector plane, and χ is defined by

$$χ ( λ , \overline{u} ) = \{ \begin{matrix}\frac{cos λ}{D ( λ ) sin λ - \overline{u} cos λ} & if \overline{w} > 0 \\ \frac{- sin λ}{D ( λ ) cos λ + \overline{u} sin λ} & if \overline{w} < 0.\end{matrix}$$

8.5 Another example of reconstruction formula which eliminates the differentiation operation with respect to the parameter λ of the well-designed general saddle curve from the reconstruction formula is given as follows:

$$f ( {x}^{->} ) = \frac{1}{4 {π}^{2}} {\int }_{0}^{2 π} ⅆ λ \frac{1}{{\overline{v}}^{2}} \int \frac{ⅆ u}{\overline{u} - u} [ {∇}_{T} {\hat{g}}_{f} ( λ , u , w ) ] {\vert}_{w = \overline{w} + κ ( u - \overline{u} )} - \frac{1}{4 {π}^{2}} {\int }_{0}^{2 π} \frac{D ( λ ) ( κ R ( λ ) - {a}_{z}^{′} ( λ ) ) - {R}^{′} ( λ ) ( \overline{w} - κ \overline{u} )}{{\overline{v}}^{2}} χ ( λ , \overline{u} ) \int ⅆ u ( \frac{\partial }{\partial w} {{g}^{̑}}_{f} ( λ , u , w ) ) {\vert}_{w = \overline{w} + κ ( u - \overline{u} )} , \\ where \\ {∇}_{T} = D ( λ ) ( R ( λ ) \frac{\partial }{\partial u} + {a}_{z}^{′} ( λ ) \frac{\partial }{\partial w} ) + {R}^{′} ( λ ) ( u \frac{\partial }{\partial u} + w \frac{\partial }{\partial w} + 1 ) .$$

9. Special Example 2 and the detailed reconstruction steps
9.1 Definition

We consider well-designed general saddles established by C-arm geometry (or ring geometry). The well-designed general saddle established by the C-arm geometry can be parameterized as (R cos α cos λ, R cos α sin λ, R sin α)^{T}, where α is a function of λ(α=α(λ)) and satisfies

$$\vert α \vert < \frac{π}{2} .$$

9.2 Detector Geometry

The coordinates for the detector are given in the reconstruction space as follows:

$$\{ \begin{matrix}{{e}^{->}}_{uc} ( λ ) = {( - sin λ , cos λ , 0 )}^{T} \\ {{e}^{->}}_{vc} ( λ ) = {( - cos αcos λ , - cos α sin λ , - sin α )}^{T} \\ {{e}^{->}}_{wc} ( λ ) = {( - sin αcos λ , - sin α sin λ , cos α )}^{T}\end{matrix}$$

The detector plane is arranged to be orthogonal to the vector \vec{e}_{vc}(λ). A projection point of the point \vec{α}(λ) onto the detector plane is the origin of the detector plane. Cartesian coordinates (u_{c},w_{c}) specify a pixel in the detector plane. The u_{c}-axis is parallel to the vector \vec{e}ᵤ, and the w_{c}-axis is parallel to the vector \vec{e}_{w}.

The distance between the detector plane and the X-ray source in the C-arm apparatus is denoted by D(>0), and the projection data on the detector plane is denoted by g_{c}(λ,u_{c},w_{c}).

9.3 Reconstruction theorem

The reconstruction theorem is performed by the following steps.

STEP 1: Filtering

FF1: Differentiation

Compute
g₁(λ,u_{c},w_{c})=∇_{c}g_{c}(λ,u_{c},w_{c})
where ∇_{c} is defined by

$${∇}_{c} = \frac{\partial }{\partial λ} + \frac{( {D}^{2} + {u}_{c}^{2} ) cos α + D {w}_{c} sin α + {u}_{c} {w}_{c} {α}^{′}}{D} \frac{\partial }{\partial {u}_{c}} + \frac{( {D}^{2} + {w}_{c}^{2} ) {α}^{′} - D {u}_{c} sin α + {u}_{c} {w}_{c} cos α}{D} \frac{\partial }{\partial {w}_{c}} .$$

FF2: Weighting
Compute

$${g}_{2} ( λ , {u}_{c} , {w}_{c} ) = \frac{D ( λ )}{\sqrt{{D}^{2} ( λ ) + {u}_{c}^{2} + {w}_{c}^{2}}} {g}_{1} ( λ , {u}_{c} , {w}_{c} ) .$$

FF3: Interpolation before filtering
g₃(λ,u_{c},z)=g₂(λ,u_{c},w_{c}(u_{c},z)),
where z denotes the parameter for the filtering plane π(z), and w_{c}(u_{c},z) is an equation of the intersection line between the filtering plane π(z) and the detector plane.
FF4: 1D Hilbert transform
Compute

$${g}_{4} ( λ , {u}_{c} , z ) = {\int }_{- ∞}^{∞} ⅆ {u}_{c}^{′} {h}_{H} ( {u}_{c} - {u}_{c}^{′} ) {g}_{3} ( λ , {u}_{c}^{′} , z ) ,$$

where h_{H} is the impulse response of the Hilbert transform.
FF5: Transform after filtering
Compute
g_{c}^{F}(λ,u_{c},w_{c})=g₄(λu_{c},z(u_{c},w_{c}))
where z(u_{c},w_{c}) denotes the parameter for the filtering plane which passes through the point (u_{c},w_{c}) on the detector plane.
STEP 2: Backprojection
Compute

$$f ( {x}^{->} ) = \frac{1}{4 π} {\int }_{0}^{2 π} ⅆ λ \frac{1}{{\overline{v}}_{c} ( {x}^{->} , λ )} {g}_{c}^{F} ( λ , {\overline{u}}_{c} ( {x}^{->} , λ ) , {\overline{w}}_{c} ( {x}^{->} , λ ) ) ,$$

where \vec{u}_{c}(\vec{x},λ),\vec{w}_{c}(\vec{x},λ) denote the coordinates of the intersection point of the detector plane and the line connecting the points \vec{x} and \vec{α}(λ), and
\vec{v}_{c}(\vec{x},λ)=R−x cos α cos λ−y cos α sin λ−z sin α.

Note that by eliminating the procedure FF5 of STEP 1 in the above reconstruction steps and incorporating the corresponding procedure in the backprojection of STEP 2, resolution in the reconstructed image can be increased.
9.4 The differentiation operation with respect to the parameter λ for the well-designed general saddle curve can be eliminated from the reconstruction steps in 9.3 in the same way as 8.4. That is, we define \vec{g}_{c} by

$${{g}^{̑}}_{c} ( λ , {u}_{c} , {w}_{c} ) = \frac{D}{\sqrt{{D}^{2} + {u}_{c}^{2} + {w}_{c}^{2}}} {g}_{c} ( λ , {u}_{c} , {w}_{c} ) .$$

Then, the reconstruction formula is given as follows:

$$f ( {x}^{->} ) = - \frac{1}{4 {π}^{2}} {\int }_{0}^{2 π} ⅆ λ \frac{DR cos α}{{\overline{v}}_{c}^{2}} \int \frac{ⅆ {u}_{c}}{{( {\overline{u}}_{c} - u )}^{2}} {\hat{g}}_{c} ( λ , {u}_{c} , {w}_{c} ) {\vert}_{{w}_{c} = {\overline{w}}_{c} + {κ}_{c} ( {u}_{c} - {\overline{u}}_{c} )} - \frac{1}{4 {π}^{2}} {\int }_{0}^{2 π} ⅆ λ \frac{DR ( {κ}_{c} cos α - {α}^{′} )}{{\overline{v}}_{c}^{2}} \int \frac{ⅆ {u}_{c}}{{\overline{u}}_{c} - {u}_{c}} ( \frac{\partial }{\partial {w}_{c}} {\hat{g}}_{c} ( λ , {u}_{c} , {w}_{c} ) ) {\vert}_{{w}_{c} = {\overline{w}}_{c} + {κ}_{c} ( {u}_{c} - {\overline{u}}_{c} )} - \frac{1}{4 {π}^{2}} {\int }_{0}^{2 π} \frac{DR ( {κ}_{c} cos α - {α}^{′} )}{{\overline{v}}_{c}^{2}} {χ}_{c} ( λ , {\overline{u}}_{c} ) \int ⅆ {u}_{c} ( \frac{\partial }{\partial {w}_{c}} {\hat{g}}_{c} ( λ , {u}_{c} , {w}_{c} ) ) {\vert}_{{w}_{c} = {\overline{w}}_{c} + {κ}_{c} ( {u}_{c} - {\overline{u}}_{c} )} \\ where {\overline{v}}_{c} ( {x}^{->} , λ ) = R - x cos α cos λ - y cos α sin λ - z sin α , \\ {\overline{u}}_{c} ( {x}^{->} , λ ) = \frac{D}{{\overline{v}}_{c} ( {x}^{->} , λ )} ( - x sin λ + y cos λ ) , \\ {w}_{c} ( {x}^{->} , λ ) = \frac{D}{{\overline{v}}_{c} ( {x}^{->} , λ )} ( - x sin α cos λ - y sin α sin λ + z cos α ) , \\ {κ}_{c} = \{ \begin{matrix}\frac{( z - R sin α ) cos λ}{sin λ ( z sin α - R )} & if z \geq R sin α \\ \frac{- ( z - R sin α ) sin λ}{cos λ ( z sin α - R )} & if z < R sin α ,\end{matrix} \\ χ ( λ , \overline{u} ) = \{ \begin{matrix}\frac{cos αcos λ}{D sin λ - {\overline{u}}_{c} cos α cos λ} & if {\overline{w}}_{0} > D tan α \\ \frac{- cos αsin λ}{D cos λ - {\overline{u}}_{c} cos αsin λ} & if {\overline{w}}_{0} < D tan α .\end{matrix}$$

9.5 Another example of formulae which eliminates the differentiation operation with respect to the parameters of the well-designed general saddle curve from the reconstruction formula is given by

$$f ( {x}^{->} ) = \frac{1}{4 {π}^{2}} {\int }_{0}^{2 π} ⅆ λ \frac{DR}{{\overline{v}}_{c}^{2}} \int \frac{ⅆ {u}_{c}}{{\overline{u}}_{c} - {u}_{c}} [ ( \begin{matrix}cos α \frac{\partial }{\partial {u}_{c}} + \\ {α}^{′} \frac{\partial }{\partial {w}_{c}}\end{matrix} ) {\hat{g}}_{c} ( λ , {u}_{c} , {w}_{c} ) ] {\vert}_{{w}_{c} = { {w}^{ \_}}_{c} + {κ}_{c} ( {u}_{c} - {{u}^{ \_}}_{c} )} - \frac{1}{4 {π}^{2}} {\int }_{0}^{2 π} \frac{DR ( {κ}_{c} cos α - {α}^{′} )}{{\overline{v}}_{c}^{2}} {χ}_{c} ( λ , {\overline{u}}_{c} ) \int ⅆ {u}_{c} ( \frac{\partial }{\partial {w}_{c}} {\hat{g}}_{c} ( λ , {u}_{c} , {w}_{c} ) ) {\vert}_{{w}_{c} = {\overline{w}}_{c} + {κ}_{c} ( {u}_{c} - {\overline{u}}_{c} )}$$

In the following, examples according to the present invention are explained.

### EXAMPLE 1

A first example of the present invention for a standard saddle given by x=R cos λ,y=R sin λ,z=h cos2λ.

The standard saddle is established by rotating an object (sample) clockwise about a rotation axis and moving the object up and down in accordance with the equation given by zₛ=−h cos2λ, where zₛ is the z coordinate of a rotation stage on which the object is placed. In this case, the X-ray source and the detector are fixed (stationary). A similar result follows by rotating the object anticlockwise. FIG. 5 shows an implementation example of a general saddle.

### EXAMPLE 2

According to the present invention, a second example for the standard saddle is given. Although the first example is easy to implement, the usage of the detector is not good. The usage of the detector is increased by moving the detector up and down in accordance with the equation given by

$${z}_{D} = \frac{D}{R} {z}_{S} ,$$

where z_{D} is the z coordinate of the detector.

### EXAMPLE 3

According to the present invention, a third example is for the C-arm system (or ring system).

There are two rotation axes in the C-arm geometry. A well-designed general saddle is easily given by restricting the value (angle) of one of the two rotation axes in the geometry by the value (angle) of the other axis.

C-arm geometry: There are two axes, λ-axis and α-axis in the C-arm system, and the apparatus has two parameters λ and α. A circle trajectory is given by fixing the angular position of the α axis and rotating the C-arm about the λ-axis. If the C-arm is rotated about the two axes, the coordinates of the X-ray source are given by (R cos αcos λ,R cos αsin λ,R sin α)^{T}.

The trajectory becomes a well-designed general saddle, if the rotation about the α-axis (α=α(λ)) satisfies certain conditions. The trajectory of the X-ray source is a well-designed general saddle, if the rotation is restricted, for example, by

$$α = α ( λ ) = k cos 2 λ , 0 < k < \frac{π}{2} , or$$

$$α = α ( λ ) = {tan}^{- 1} \frac{k cos 2 λ}{\sqrt{1 - {k}^{2}}} , 0 < k < 1.$$

Refer to FIG. 7 showing the C-arm geometry.

The well-defined general saddle can be realized also in the ring geometry instead of the C-arm geometry. The relative positional relationship between the X-ray source and the detector (detector plane) on the saddle trajectory is the same.

### EXAMPLE 4

According to the present invention, a fourth example is given. The general saddle CT apparatus can reconstruct a thin (planar) object more accurately than oblique CT by avoiding directions along which the X-ray hardly penetrates the object, although the magnification cannot be increased so much. The reason can be explained by the following three aspects:

(1) The general saddle trajectory is capable of penetrating the object in most of the directions as the oblique CT can.

(2) In the case of the general saddle, projection data obtained at a most slanted (lowest) angle can be utilized.

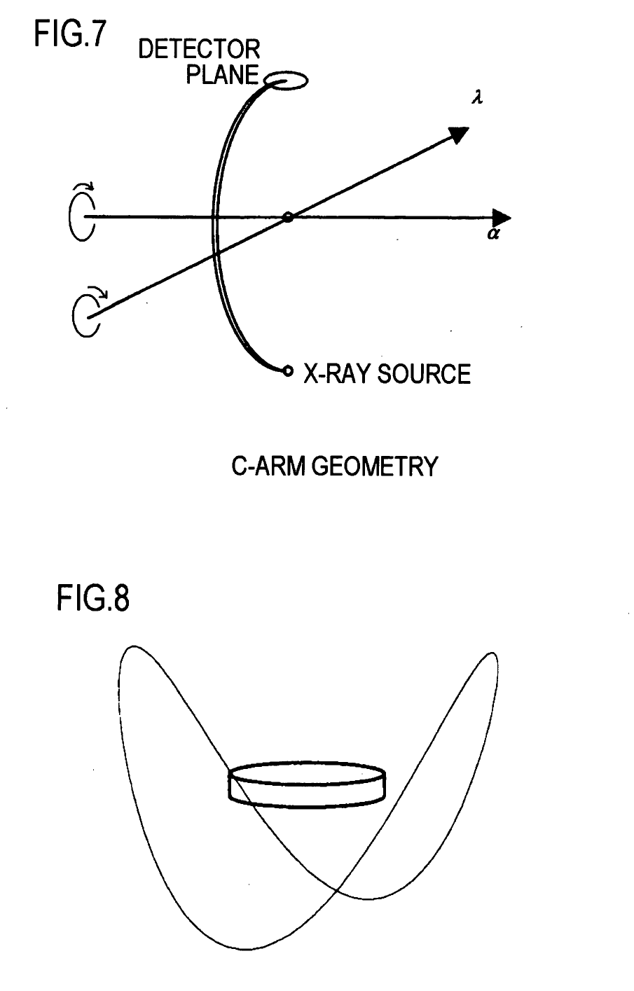

(3) The projection data of those projections which do not penetrate the object can be neglected and need not be used in reconstruction. Although some data lack in such a case, their effect is less severe for the general saddle CT and more accurate images can be reconstructed than the oblique CT. Refer to FIG. 8 showing the geometry for reconstructing a thin (planar) object with a general saddle.

### EXAMPLE 5

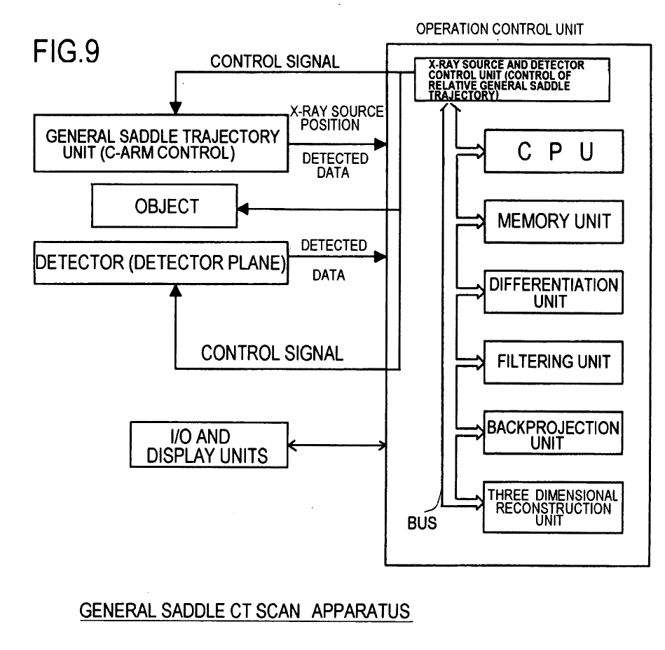

According to the present invention, a fifth example is given. FIG. 9 shows a block diagram of an apparatus for performing a method according to the present invention. A general saddle CT scan apparatus comprises a general saddle trajectory unit (for example, a dividing control unit for a C-arm system or ring system), a detector with a detector plane, an operation control unit which receives detected data from these units and controls them, and a I/O and display unit. The operation control unit further comprises an X-ray source-detector system control unit, a CPU, a memory unit (which may include ROM, RAM, register and hard disk etc.), a differentiating unit, a filtering unit, backprojection unit, and a three dimensional (3D) reconstruction unit, all of which are connected to a bus. The operation unit comprises also an input/output interface and other commodity parts, which are not shown in the figure.

The differentiating, filtering, backprojection, and 3D reconstruction units may be implemented by both software and/or hardware. Although not shown in the figure, each detailed step of the embodiment can be performed one by one correspondingly with these units. Each step of the method or operation steps according to the present invention memorizes projection data (detected data) upon needs and transfer the data to the next step.

Equations used for each step is memorized in a ROM beforehand, or memorized, if necessary, in a hard disk or other high-speed memory and read out of it. A clock signal (not shown in the figure) controls the total system. First of all, a general saddle is specified and set through the I/O and display units.

After an object to be measured (sample) is placed at a predetermined position of the apparatus (for example, the center of a C-arm system), the initial position of the X-ray source is placed at a point a (λ) on the predetermined general saddle trajectory. After a detector plane of the detector is placed to satisfy predetermined conditions, an X-ray cone beam is irradiated onto the sample under control. The projection (image) which penetrates the object is detected on the detector plane of the detector and the detected data is memorized in accordance with the coordinate system of the detector or the detector plane. The data detected by the coordinate system of the detector plane is filtered along an intersection line of a predetermined filtering plane and the detector plane. A predetermined process of each step is performed on the data by each processing unit before and after the filtering process. The data generated in each step is memorized in the memory and forwarded to a subsequent step.

The data after backprojection is memorized and stored as a basic data to reconstruct 3D images. The data is reconstructed to form a 3D image through the 3D reconstruction unit and, if necessary, displayed on the display using a visualization program. These operations are formed by CPU. The control signals are sent from CPU through I/O circuits (not shown in the figure), and the detection signals are output from the detector. The positions of the X-ray source-detector system are also detected and utilized (fed back) to control their relative position.

According to the principle of present invention, there is provided a CT method which makes it possible to process CT slice images one by one. The CT method can be most easily implemented and reduces the computational time significantly. Each processing (slice) image is memorized in the memory device in association with the parameter λ which specifies the location. Then the process proceeds to the next scanning step (the next reconstruction step). In the same way, the steps are repeated along the general saddle trajectory. When the X-ray source returns to the initial position, one cycle is completed.

Since one cycle of the scan ends in a short time, X-ray exposure of an object during one CT scan is reduced. There is also provided a four dimensional CT with time as a parameter in principle, because one cycle of the CT scan ends in a short time and the X-ray source returns to the initial position on the general saddle trajectory after one cycle. Therefore, a dynamic four dimensional CT which operates online in the time sequence is made possible by the present invention, which brings revolution in the CT scan for moving objects such as heart (walls).

### EXAMPLE 6

(Simulation Example 1)

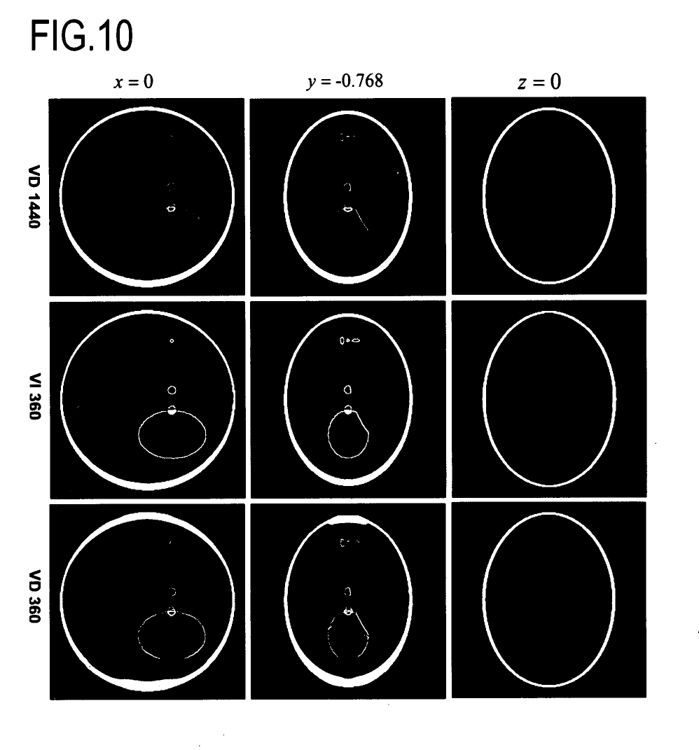

According to the present invention, there is provided a sixth example for simulation use. The simulation studies have been done for the Shepp-Logan phantom with the view differencing (VD) method and the view independent (VI) method based on general saddles according to (8.3) and (8.4) of the present invention, respectively. FIG. 10 shows cross sections of the reconstructed 3D images by each method at x=0, y=−0.768 cm, and z=0.

In FIG. 10, VD1440 denotes the reconstructed images by the VD algorithm according to (8.3) of the present invention taken from projections in the (1440) direction. VI 360 denotes the reconstructed images by the VI algorithm according to (8.4) of the present invention taken from projections in the (360) direction. In the same way, VD 360 denotes the reconstructed images by the VD algorithm according to (8.3) of the present invention taken from projections in the (360) direction.

### EXAMPLE 7

(Simulation Example 2)

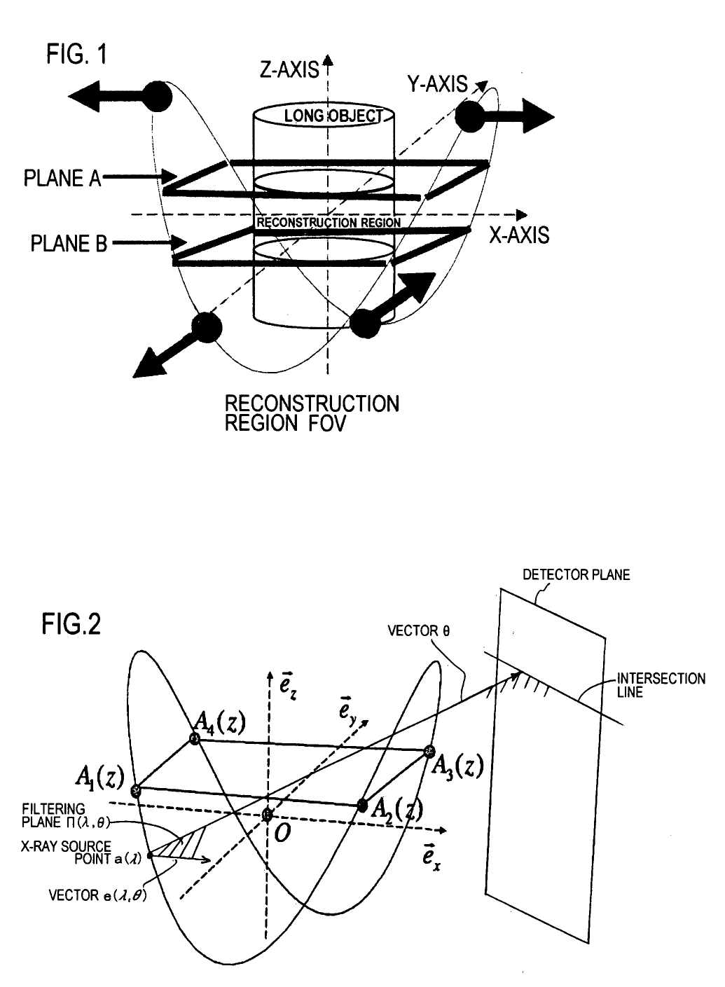

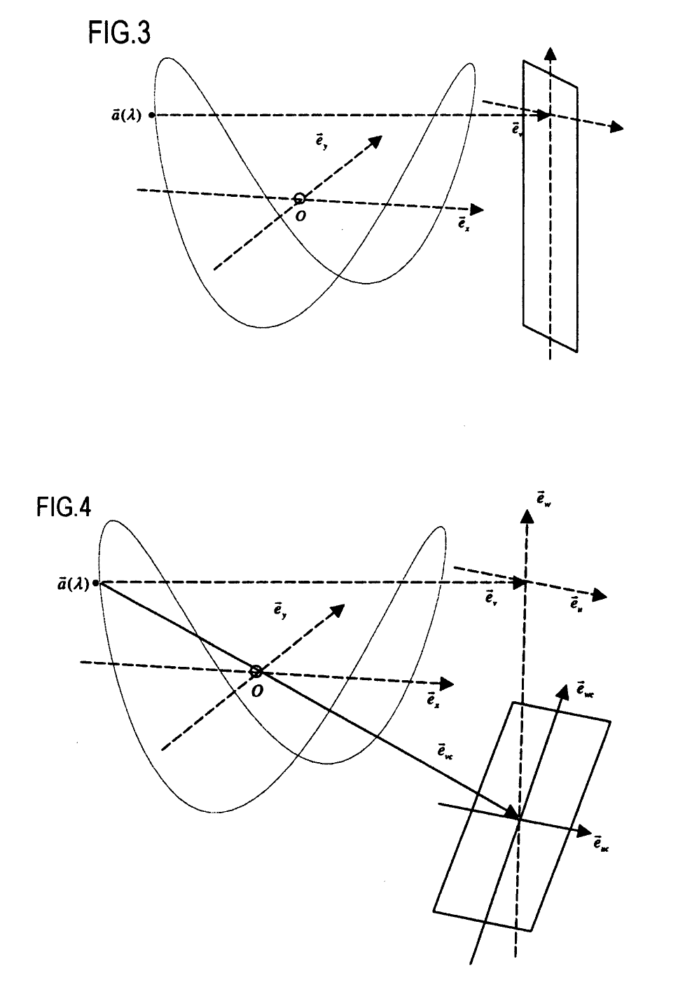

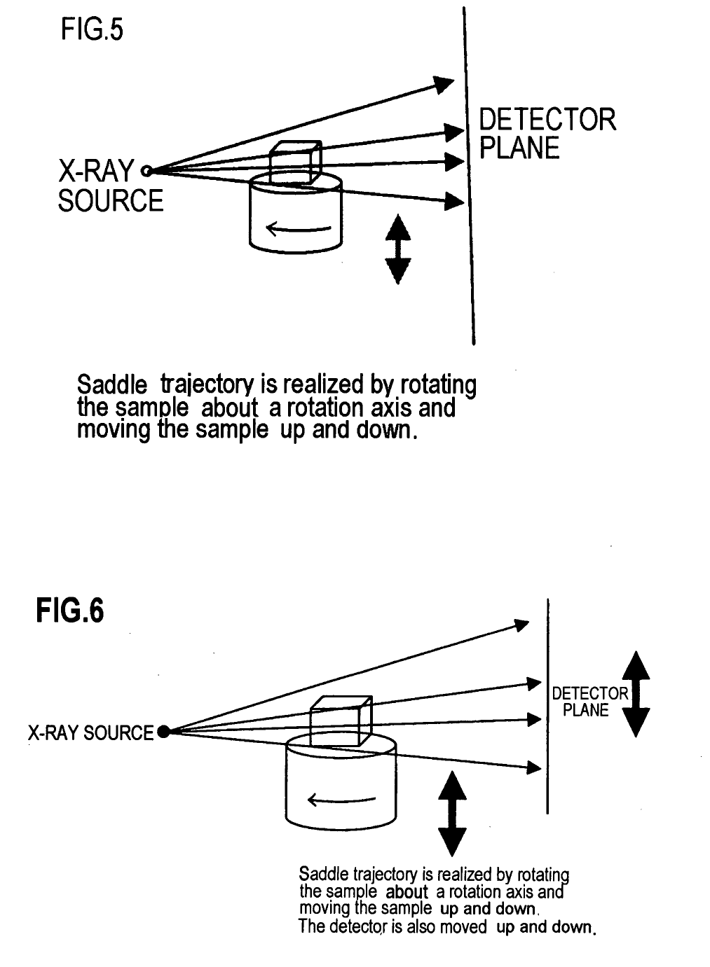

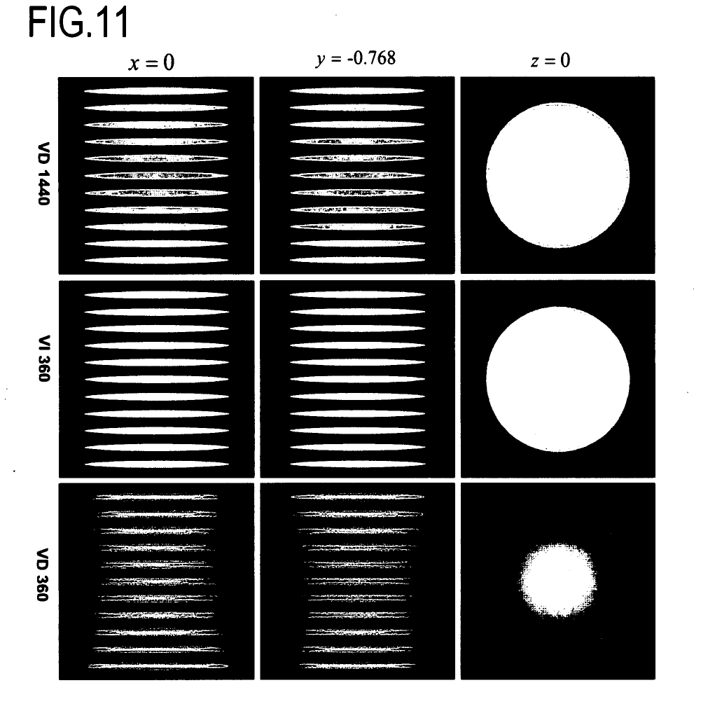

According to the present invention, there is provided a seventh example for simulation use. The simulation studies have been done for a disc phantom with the view differencing (VD) method and the view independent (VI) method based on a general saddle trajectory according to the (8.3) and (8.4) of the present invention, respectively. FIG. 1 shows cross sections (slices) of the reconstructed 3D images by each method at x=0, y=−0.768 cm, and z=0. In FIG. 11, VD1440 denotes the reconstructed images by the VD algorithm according to (8.3) of the present invention taken from projections in the direction of (1440). VI 360 denotes the reconstructed images by the VI algorithm according to (8.4) of the present invention taken from projections in the (360) direction. In the same way, VD 360 denotes the reconstructed images by the VD algorithm according to (8.3) of the present invention taken from projections in the (360) direction.

### EXAMPLE 8

(Simulation Example 3)

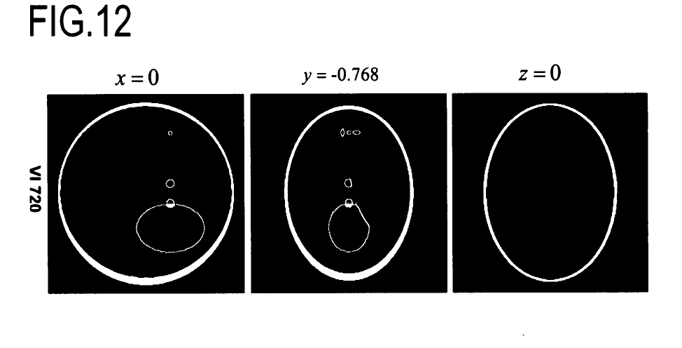

According to the present invention, there is provided an eighth example for simulation use. The simulation studies have been done for the Shepp-Logan phantom with the view independent (VI) method based on a well-designed general saddle trajectory established by a C-arm geometry system according to (9.4) of the present invention. FIG. 12 shows cross sections of the reconstructed 3D images by each method at x=0, y=−0.768 cm, and z=0. In FIG. 12, VI 720 denotes the reconstructed images by the VI algorithm according to (9.4) of the present invention taken from projections in the (720) direction.

The meritorious effects of the present invention are summarized as follows.

According to the present invention, a general saddle scan method is proposed for CT imaging of a short object as well as partial CT imaging of a long object. Since the general saddle satisfies the cone-beam CT data completeness condition, the general saddle CT can reconstruct the object more accurately than the conventional cone-beam CT of the circular trajectory. Moreover, since the general saddle scan avoids the problem of overscan, it requires shorter measurement time and less X-ray exposure of objects than the helical scan. The general saddle scan method according to the present invention also makes it possible to scan an object serially along with time, because the relative position between the X-ray source and the object returns to the original position after one rotation (cycle). Since the general saddle proposed in the present invention has wider applicability than the conventional saddles, it can be used in many easily realizable CT scan methods.

It should be noted that there had been no simple and effective reconstruction algorithm even for the simplest saddle trajectory before the present invention. The present invention provides a filtered backprojection (FBP)-type reconstruction algorithm for general saddles for the first time. Since the proposed algorithm is of the FBP-type, it is easy to implement and the computational speed is also accelerated.

The step of identifying a set of π-lines (according to the π-line based methods: Pack et al 2004, 2005, Xia et al 2005, Yu et al 2005, Zou et al 2005) is not necessary to implement the proposed algorithms in the present invention. The exact FBP reconstruction algorithms are proposed for the general saddle. The key idea of the proposed algorithm is that the filtering plane is parallel to one of the two horizontal axes which define the general saddle. The proposed algorithms are theoretically exact, have a shift-invariant FBP structure, and do not depend from the concept of π-line.

[Applicable Industrial Fields]

The present invention triggers the use of the general saddle as a scan method for industrial inspection and medical diagnostics. Since a point on the general saddle returns to the original position after one rotation (cycle), serial scan of an object is also possible. Therefore, the general saddle is applicable to the following fields for examples.

1. Four dimensional cardiac CT (e.g., medical use)

2. Automatic inspection apparatus (e.g., industrial use)

The disclosures of articles of H. Yang, M. Li, K. Koizumi and H. Kudo entitled “Exact cone beam reconstruction for a saddle trajectory” (Phys. Med. Biol. 51 (2006) 1157-1172), and “View-independent reconstruction algorithms for cone beam CT with general saddle trajectory”, Phys. Med. Biol. 51 (2006) 3865-3884 are incorporated herein by reference thereto for further detail and developments of the principles of the present invention in the theoretical aspects. The articles are coauthored by the present inventors and both published after the priority date of the present invention.

It should be noted that other objects, features and aspects of the present invention will become apparent in the entire disclosure and that modifications may be done without departing the gist and scope of the present invention as disclosed herein and claimed as appended herewith.

Also it should be noted that any combination of the disclosed and/or claimed elements, matters and/or items may fall under the modifications aforementioned.

What is claimed is:

1. A general saddle CT scan method comprising:
(a) placing an object in association with a general saddle curve defined by the following conditions; and
(b) moving an X-ray source relatively to the object along the general saddle curve and detecting X-ray projection data of the object projected on a detector plane;
wherein said saddle curve is a simple smooth curve which is single-connected, bounded and closed satisfies the following conditions against a family of continuous parallel planes in a three dimensional space:
(1) for any plane in the family of parallel planes, there exist four different intersection points between the plane and the closed curve;
(2) said four intersection points form a rectangle; and
(3) rectangles obtained for any two different planes in the family of parallel planes are parallel to each other.

2. A general saddle CT scan and reconstruction method comprising:
(a) placing an object in association with a general saddle curve defined by the following conditions;
(b) moving an X-ray source relatively to the object along the general saddle curve and detecting X-ray projection data of the object projected on a detector plane;
(c) filtering the projection data along a predetermined filtering plane which is parallel to one of two major axes of the general saddle curve; and
(d) performing three-dimensional reconstruction of the object through backprojection,
wherein said general saddle curve is a simple smooth curve which single-connected, bounded and closed satisfies the following conditions against a family of continuous parallel planes in a three dimensional space:
(1) for any plane in the family of parallel planes, there exist four different intersection points between the plane and the closed curve;
(2) said four intersection points form a rectangle; and
(3) rectangles obtained for any two different planes in the family of parallel planes are parallel to each other.

3. The general saddle CT scan and reconstruction method according to claim 2, wherein the filtering is performed along an intersection line between said filtering plane and the detector plane.

4. The general saddle CT scan and reconstruction method according to claim 2, wherein the filtering plane Π(λ,\vec{θ}) is chosen to satisfy the following conditions in relation to a point \vec{α}(λ) of the X-ray source on the general saddle curve:
(1) the filtering plane passes through the point \vec{α}(λ);
(2) the filtering plane is parallel to a vector \vec{θ}=(θₓ,θ_{y},θ_{z})^{T} extending from the point \vec{α}(λ) to a point on the detector plane; and
(3) the filtering plane is parallel to a vector \vec{e}(λ,\vec{θ}), where the vector \vec{e}(λ,\vec{θ}) is chosen to satisfy the following condition:
$${e}^{->} ( λ , {θ}^{->} ) = \{ \begin{matrix}{{e}^{->}}_{x} if {θ}_{z} \geq 0 \\ {{e}^{->}}_{y} if {θ}_{z} < 0.\end{matrix}$$

5. The general saddle CT scan and reconstruction method according to claim 2, wherein the filtering is performed by
$${g}_{F} ( λ , {θ}^{->} ) = {\int }_{- π}^{π} ⅆ γ \frac{1}{sin γ} \frac{\partial }{\partial \overline{λ}} p ( \overline{λ} , ξ ( λ , θ , γ ) ) {\vert}_{\overline{λ} = λ} , θ ∈ {S}^{2}$$
$$where$$
$${ξ}^{->} ( λ , {θ}^{->} , γ ) = cos γ {θ}^{->} + sin γ \frac{{e}^{->} ( λ , {θ}^{->} ) - ( {e}^{->} ( λ , {θ}^{->} ) \cdot {θ}^{->} ) {θ}^{->}}{\vert {e}^{->} ( λ , {θ}^{->} ) - ( {e}^{->} ( λ , {θ}^{->} ) \cdot {θ}^{->} ) {θ}^{->} \vert} ,$$
and g(λ,\vec{ξ}) denotes the projection data.

6. The general saddle CT scan and reconstruction method according to claim 2, wherein the backprojection is performed by
$$f ( {x}^{->} ) = - \frac{1}{4 {π}^{2}} {\int }_{0}^{2 π} \frac{ⅆ λ}{\vert {x}^{->} - {a}^{->} ( λ ) \vert} {g}_{F} ( λ , \frac{{x}^{->} - {a}^{->} ( λ )}{\vert {x}^{->} - {a}^{->} ( λ ) \vert} ) ,$$
where f(\vec{x}) denotes CT value at each point of the object.

7. The general saddle CT scan method according to claim 1, wherein a well-designed general saddle curve defined in the following is employed as the general saddle curve in case where the object is long, and the reconstruction region FOV is sandwiched between two (horizontal) planes crossing the object, the general saddle curve being termed “well-designed” subject to the following conditions:
(1) left (or right) part of a given general saddle curve above a first (horizontal) plane is located on the left (or right) side of the reconstruction region FOV; and
(2) front (or back) part of a given general saddle curve below a second (horizontal) plane is located on the front (or back) side of a front (or back) plane of the reconstruction region FOV.

8. The general saddle CT scan and reconstruction method according to claim 4, wherein a three-dimensional reconstruction of the object is performed by repeating the steps (c) and (d) for each value of the parameter λ of the general saddle curve.

9. The general saddle CT scan and reconstruction method according to claim 4, wherein the filtering comprises:
differentiation of the projection data with respect to the parameter λ of the general saddle curve;
weighting after the differentiation;
interpolation before filtering; and
1D Hilbert transform of the interpolated data.

10. The general saddle CT scan and reconstruction method according to claim 9, wherein filtering and coordinate transformation of the filtered data are performed after the 1D Hilbert transform.

11. The general saddle CT scan and reconstruction method according to claim 9, wherein the backprojection is performed using filtering and coordinate transformation of the filtered data after the 1D Hilbert transform.

12. The general saddle CT scan and reconstruction method according to claim 4, wherein the filtering is performed by defining two weighted data based on the projection data, and the reconstruction is performed using these data without differentiation operation with respect to the parameter λ of the general saddle curve.

13. The general saddle CT scan method according to claim 1, wherein a well-designed general saddle curve is produced by restricting value about one axis among two rotation axes in C-arm geometry or ring geometry with respect to a value of the other axis.

14. The general saddle CT scan method according to claim 1, wherein the general saddle curve is established by rotating the object about a rotation axis thereof, fixing either one of the X-ray source and the detector plane, and controlling their relative positional relationship.

15. The general saddle CT scan method according to claim 1, wherein the general saddle curve is established by fixing the X-ray source and the detector plane, rotating the object about its rotation axis and moving the object forward and reverseward along a direction of its rotation axis in accordance with a predetermined equation.

16. A computer program product for executing the method of claim 1 by a computer.

17. A general saddle CT scan apparatus comprising:
(a) a support unit which supports an object relatively to a general saddle curve defined by the conditions below;
(b) a driving unit for an X-ray source-object-detector system, which provides relative movement of an X-ray source and a detector plane along a general saddle curve relatively to the object; and
(c) a capture unit which detects projection data of X-ray radiated from the X-ray source and penetrated through the object;
wherein said general saddle curve is defined as such if a simple smooth curve which is single-connected, bounded and closed satisfies the following conditions against a family of continuous parallel planes in a three dimensional space:
(1) for any plane in the family of parallel planes, there exist four different intersection points between the plane and the closed curve;
(2) said four intersection points form a rectangle; and
(3) rectangles obtained for any two different planes in the family of parallel planes are parallel to each other.

18. A general saddle CT scan apparatus comprising:
(a) a support unit which supports an object relatively to a general saddle curve defined by the conditions below;
(b) a driving unit for an X-ray source-object-detector system, which provides relative movement of an X-ray source and a detector plane along a general saddle curve relatively to the object;
(c) a capture unit which detects projection data of X-ray radiated from the X-ray source and penetrated through the object;
(d) a filtering unit that filters the projection data along a predetermined filtering plane which is parallel to one of two major axes of the general saddle curve; and
(e) a back projection and reconstruction unit that performs reconstruction of the object through backprojection;
wherein said general saddle curve is defined as such if a simple smooth curve which is single-connected, bounded and closed satisfies the following conditions against a family of continuous parallel planes in a three dimensional space:
(1) for any plane in the family of the parallel planes, there exist four different intersection points between the plane and the closed curve;
(2) said four intersection points form a rectangle; and
(3) rectangles obtained for any two different planes in the family of parallel planes are parallel to each other.

19. The general saddle CT scan apparatus according to claim 17, wherein the filtering unit performs the filtering along an intersection line of a filtering plane and a detector plane.

20. The general saddle CT scan apparatus according to claim 17, wherein a filtering plane Π(λ,\vec{θ}) is chosen to satisfy the following conditions in relation a point of the X-ray source \vec{α}(λ) on the general saddle curve:
(1) the filtering plane passes through the point \vec{α}(λ);
(2) the filtering plane is parallel to a vector \vec{θ}=(θₓ,θ_{y},θ_{z})^{T} extending from the point \vec{α}(λ) to a point on the detector plane; and
(3) the filtering plane is parallel to a vector \vec{e}(λ,\vec{θ}),
where the vector \vec{e}(λ,\vec{θ}) is chosen to satisfy the following condition:
$${e}^{->} ( λ , {θ}^{->} ) = \{ \begin{matrix}{{e}^{->}}_{x} if {θ}_{z} \geq 0 \\ {{e}^{->}}_{y} if {θ}_{z} < 0.\end{matrix}$$

21. The general saddle CT scan apparatus according to claim 18, wherein the filtering is performed by
$${g}_{F} ( λ , {θ}^{->} ) = {\int }_{- π}^{π} ⅆ γ \frac{1}{sin γ} \frac{\partial }{\partial \overline{λ}} p ( \overline{λ} , ξ ( λ , θ , γ ) ) {\vert}_{\overline{λ} = λ} , θ ∈ {S}^{2}$$
where
$${ξ}^{->} ( λ , {θ}^{->} , γ ) = cos γ {θ}^{->} + sin γ \frac{{e}^{->} ( λ , {θ}^{->} ) - ( {e}^{->} ( λ , {θ}^{->} ) \cdot {θ}^{->} ) {θ}^{->}}{\vert {e}^{->} ( λ , {θ}^{->} ) - ( {e}^{->} ( λ , {θ}^{->} ) \cdot {θ}^{->} ) {θ}^{->} \vert} ,$$
and g(λ,\vec{ξ}) denotes the projection data.

22. The general saddle CT scan apparatus according to claim 18, wherein the backprojection is performed by
$$f ( {x}^{->} ) = - \frac{1}{4 {π}^{2}} {\int }_{0}^{2 π} \frac{ⅆ λ}{\vert {x}^{->} - {a}^{->} ( λ ) \vert} {g}_{F} ( λ , \frac{{x}^{->} - {a}^{->} ( λ )}{\vert {x}^{->} - {a}^{->} ( λ ) \vert} ) ,$$
where f(\vec{x}) denotes CT value at each point of the object.

23. The general saddle CT scan apparatus according to claim 18, wherein a well-designed general saddle curve defined in the following is employed as the general saddle curve, in case where the object is long, and the reconstruction region FOV is sandwiched between two (horizontal) planes crossing the object, the general saddle curve being termed “well-designed” subject to the following conditions:
(1) left (or right) part of a given general saddle curve above a first (horizontal) plane is located on the left (or right side) of the reconstruction region FOV; and
(2) front (or back) part of a given general saddle curve below a second (horizontal) plane is located on the front (back) side of a front (or back) plane of the reconstruction region FOV.

24. The general saddle CT scan apparatus according to claim 18, further comprising a memory unit that stores the backprojection and reconstruction data from the back projection and reconstruction unit (e); and
a three-dimensional reconstruction unit that reconstructs the object from the pack projection and reconstruction data.

25. The general saddle CT scan apparatus according to claim 17, wherein the well-designed general saddle curve is produced by restricting value about one axis among two rotation axes in C-arm geometry or ring geometry with respect to a value of another axis.

26. The general saddle CT scan apparatus according to claim 17, wherein the general saddle curve is established by rotating the sample about a rotation axis thereof, fixing either one of the X-ray source and the detector plane, and controlling their relative positional relationship.

27. The general saddle CT scan apparatus according to claim 17, wherein the general saddle curve is established by fixing the X-ray source and the detector plane, rotating the sample about a rotation axis and moving the object forward and reverseward along a direction of its rotation axis in accordance with a predetermined equation.
